(19) United States
(12) Patent Application Publication
(10) Pub. No.: US 2014/0171895 A1
(43) Pub. Date: Jun. 19, 2014

(54) Laminate and Process For Making Same

(71) Applicant: KIMBERLY-CLARK WORLDWIDE, INC., Neenah, WI (US)

(72) Inventors: Oomman P. Thomas, Alpharetta, GA (US); Peter J. Allen, Neenah, WI (US); Vasily A. Topolkaraev, Appleton, WI (US); Jenny Day, Woodstock, GA (US); Francis P. Abuto, Johns Creek, GA (US); Jerome J. Schwalen, Marietta, GA (US)

(73) Assignee: KIMBERLY-CLARK WORLDWIDE, INC., Neenah, WI (US)

(21) Appl. No.: 13/720,194

(22) Filed: Dec. 19, 2012

(51) Int. Cl. B32B 7/04; A61F 13/49

(52) U.S. Cl. CPC B32B 7/045; A61F 13/49012
USPC 604/385.3; 428/43; 2/69; 2/455; 2/171; 36/72 R; 156/221

(57) ABSTRACT

Laminates are described that contain a frangible layer adhered to at least one extensible layer, such as an elastic layer. In one embodiment, a frangible layer is positioned between two opposing elastic layers. The frangible layer includes lines of separation that generally extend in a first direction. The lines of separation allow the elastic layers to stretch and recover in a direction perpendicular or skew to the lines of separation. In one particular embodiment, the lines of separation comprise lines where the frangible layer has been weakened. The lines of separation can be formed after the laminate is made using groove rolls.

### BACKGROUND

Laminates that have elongation properties are used in numerous and diverse applications. For instance, an elastic laminate generally refers to a multi-layered material that has elastic properties. An elastic laminate, for instance, can be stretched and, once the stretching force is removed, the material retracts and recovers. Some elastic laminates, such as elastic laminates used in clothing, are intended to be used over and over. Other elastic laminates, however, are intended to be disposable after a single use. For instance, absorbent articles such as diapers, diaper pants, child training pants including pull-on versions, feminine hygiene products, adult incontinence products, and the like typically include elastic laminates that are positioned on the article and are intended to optimize fit, make the article more comfortable to wear through improved fit, and/or improve the ability of the article to absorb liquids while preventing leakage through improved containment flaps and gasketing.

In one embodiment, elastic laminates are made by attaching a facing to an elastic film or to elastic filaments. For example, the elastic film or filament can be stretched and then bonded to the facing which causes the facing to gather. In this manner, a non-elastic facing can be attached to an elastic film or filament while still allowing the elastic film or filament to be stretched to a certain degree.

In another embodiment, an elastic laminate can be produced by attaching a stretchable and elastomeric ply to a second ply that is elongatable. The elongatable ply, upon stretching of the laminate, will be at least to a degree permanently elongated so that, upon release of the applied tensile forces, it will not return to its original undistorted configuration. Such structures are disclosed, for instance, in at least one of U.S. Pat. No. 5,143,679, U.S. Pat. No. 5,167,897, U.S. Pat. No. 5,156,793, and U.S. Pat. No. 5,518,801, which are all incorporated herein by reference. Unfortunately, however, the ply that is elongatable can have a tendency to restrict the amount the elastomeric ply can be stretched.

One reoccurring problem in the art is the ability to produce an elastic laminate that has a lofty feel, especially when stretched. Lofty materials are generally preferred by consumers and are perceived to have greater softness and provide greater comfort. When attaching gathered layers to elastic materials, however, the gathered layers have a tendency to restrict the elastic properties of the resulting laminate. These materials can also be very difficult and expensive to produce since, in some embodiments, the elastic layer is in a stretched state when attached to a material that gathers when the elastic layer is relaxed. Simply increasing the thickness of the elastic layers can also cause various problems. Increasing the thickness of the elastic layer, for instance, not only increases material costs but also produces laminates that are generally more difficult to stretch.

In view of the above, a need exists for an elastic laminate with increased loft characteristics without adversely impacting the elastic properties of the laminate. A need also exists for an elastic laminate that has high loft at a relatively low basis weight.

### DEFINITIONS

As used herein, the term “breathable” means pervious to water vapor and gases. In other words, “breathable barriers” and “breathable films” allow water vapor to pass therethrough. For example, “breathable” can refer to a film or laminate having water vapor transmission rate (WVTR) of at least about 300 g/m²/24 hours measured using ASTM Standard E96-80, upright cup method, with minor variations as described in the following Test Procedure.

A measure of the breathability of a fabric is the water vapor transmission rate (WVTR) which, for sample materials, is calculated essentially in accordance with ASTM Standard E96-80 with minor variations in test procedure as set forth hereinbelow. Circular samples measuring three inches in diameter are cut from each of the test materials, and tested along with a control, which is a piece of “CELGARD” 2500 sheet from Celanese Separation Products of Charlotte, N.C. “CELGARD” 2500 sheet is a microporous polypropylene sheet. Three samples are prepared for each material. The test dish is a No. 60-1 Vapometer pan distributed by Thwing-Albert Instrument Company of Philadelphia, Pa. 100 milliliters of water is poured into each Vapometer pan and individual samples of the test materials and control material are placed across the open tops of the individual pans. Screw-on flanges are tightened to form a seal along the edges of the pan, leaving the associated test material or control material exposed to the ambient atmosphere over a 6.5 cm diameter circle having an exposed area of approximately 33.17 square centimeters. The pans are placed in a forced air oven at 100° F. (32° C.) for one hour to equilibrate. The oven is a constant temperature oven with external air circulating through it to prevent water vapor accumulation inside. A suitable forced air oven is, for example, a Blue M Power-O-Matic 600 oven distributed by Blue M Electric Company of Blue Island, Ill. Upon completion of the equilibration, the pans are removed from the oven, weighed and immediately returned to the oven. After 24 hours, the pans are removed from the oven and weighed again. The preliminary test water vapor transmission rate values are calculated as follows: Test WVTR′(grams weight loss over 24 hours)×(315.5 g/m²/24 hours).

The relative humidity within the oven is not specifically controlled. Under predetermined set conditions of 100° F. (32° C.) and ambient relative humidity, the WVTR for the “CELGARD” 2500 control has been defined to be 5000 grams per square meter for 24 hours. Accordingly, the control sample was run with each test and the preliminary test values were corrected to set conditions using the following equation:

WVTR′(test WVTR/control WVTR)×(5000 g/m²/24 hrs.)

As used herein, the term “filament” refers to a type of fiber that is described as a continuous strand that has a large ratio of length to diameter, such as, for example, a ratio of 1000 or more.

As used herein, “meltblown fibers” refers to fibers formed by extruding a molten thermoplastic material through a plurality of fine, usually circular, die capillaries as molten threads or filaments into converging high velocity, usually hot, gas (e.g. air) streams which attenuate the filaments of thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited on a collecting surface.

As used herein, the term “nonwoven web” refers to a web having a structure of individual fibers or threads which are interlaid, but not in an identifiable manner as in a knitted fabric. Nonwoven webs or fabrics have been formed from many processes, such as, for example, meltblowing processes, spunbonding processes, and bonded carded web processes. Paper webs and tissue webs, as used herein, are considered nonwoven webs. The basis weight of nonwoven fabrics is usually expressed in ounces of material per square yard (osy) or grams per square meter (gsm) and the fibers diameters are usually expressed in microns, (Note that to convert from osy to gsm, multiply osy by 33.91).

As used herein, “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992 to Kinney, U.S. Pat. No. 3,341,394 to Kinney, U.S. Pat. No. 3,502,763 to Hartman, and U.S. Pat. No. 3,542,615 to Dobo et al. Spunbond fibers are generally not tacky when they are deposited on a collecting surface. Spunbond fibers are generally continuous and have average diameters (from a sample of at least 10) larger than 7 microns, and more particularly, between about 10 and 40 microns.

“Elastic” and “elastomeric” refer to a material or composite which can be elongated by at least 25 percent of its relaxed length and which will recover, upon release of the applied force, at least 10 percent of its elongation. It is generally desirable that the elastomeric material or composite be capable of being elongated by at least 100 percent, more desirably by at least 300 percent, of its relaxed length and recover, upon release of an applied force, at least 50 percent of its elongation.

“Extensible” refers to a material or composite which can be elongated by at least 25% of its relaxed length. For instance, the material can be elongated in certain embodiments by at least 100%, by at least 300%, by at least 400%, by at least 500%, or by at least 600% in one direction prior to breaking (according to ASTM Test D882). An extensible layer or laminate can be elastic or non-elastic.

“Film” refers to a thermoplastic film made using a film extrusion and/or foaming process, such as a cast film or blown film extrusion process. The term includes apertured films, slit films, and other porous films which constitute liquid transfer films, as well as films which do not transfer liquid.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact.

“Machine direction” refers to the length of a fabric in the direction in which the fabric is produced, as opposed to “cross direction” which refers to a direction generally perpendicular to the machine direction.

“Polymers” include, but are not limited to, homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic symmetries.

### SUMMARY

in general, the present disclosure is directed to a laminate that has elongation properties. The laminate made according to the present disclosure is at least extensible. In one embodiment, the laminate is extensible but non-elastic. In an alternative embodiment, the laminate comprises an elastic laminate. The laminate includes at least two layers. In accordance with the present disclosure, a frangible layer is attached to an elastic layer. The frangible layer includes a plurality of lines of separation that extend in at least one direction. The lines of separation, for instance, can comprise lines where the frangible layer is weakened, such as by being partially or completely severed. In this manner, when the laminate is stretched in a direction skew or perpendicular to the lines of separation, the elastic layer can be stretched and allowed to recover without interference from the frangible layer. The frangible layer, on the other hand, can provide numerous and diverse benefits to the laminate. For instance, the frangible layer can increase the loft characteristics of the laminate. In another embodiment, the frangible layer can provide strength, rigidity, or other functional advantages.

In one embodiment, for instance, the present disclosure is directed to an elastic laminate that includes a frangible layer positioned in between a first elastic layer and a second elastic layer. Each elastic layer may comprise, for instance, a film, such as a multi-layered film. The frangible layer, on the other hand, may comprise a nonwoven material such as a paper web, a meltspun web or a bonded carded web, a film, or a foil. The elastic laminate includes a first direction and a second direction. In accordance with the present disclosure, the frangible layer includes lines of separation that extend in at least the first direction. The lines of separation decouple the frangible layer from the elastic layer at least in localized and distinct areas allowing the elastic layer to stretch and recover along the second direction. As used herein, the term “decouple” means that the extensible layer may stretch without being substantially limited by the frangible layer in at least one direction. In other words, the lines of separation allow the extensible layer to be stretched in at least one direction without being bound by the stretch limitations of the frangible layer.

In one embodiment, the lines of separation comprise lines that are generally weaker than the remainder of the frangible layer. The lines of separation, for instance, may comprise lines where the layer has been completely severed or partially severed. In an alternative embodiment, the lines of separation may comprise lines of lower basis weight in the layer. In still another embodiment, the lines of separation may comprise lines of embossment. The lines of separation can have any suitable construction such that the frangible layer will separate when stretched with the elastic layers.

In one embodiment, for instance, the lines of separation are formed in the frangible layer after the frangible layer has been positioned in between the first elastic layer and the second elastic layer. For instance, in one embodiment, the laminate comprising the three layers is fed in between a first roller and a second roller wherein at least one of the rollers defines grooves. The laminate is fed in between the two rollers with sufficient nip pressure to form the lines of separation in the frangible layer without cutting the first elastic layer or the second elastic layer.

In one embodiment, the frangible layer is much less elastic than the elastic layers. For instance, the frangible layer may have an elongation to break that is less than 75%, such as less than 80%, such as less than 85%, such as less than 90%, such as even less than 95% than the elongation of break of one of the elastic layers. Elongation at break can be measured according to ASTM Test D-638-02,

As described above, the frangible layer, in one embodiment, may comprise a nonwoven material, The nonwoven material may comprise a spunbond web, a meltblown web, a hydroentangled web, a coform web or a bonded carded web. In one embodiment, the nonwoven material comprises a tissue web. The tissue web can have a macroscopic pattern of ridges and valleys which provides greater loft to the laminate. The tissue web can contain exclusively pulp fibers or may contain pulp fibers combined with synthetic fibers.

The elastic layers may comprise monolayer films or multi-layered films. In one embodiment, at least one of the elastic layers may be perforated to allow the frangible layer to absorb liquids. In fact, in one embodiment, the frangible layer may contain absorbent particles, such as superabsorbent particles.

In addition to the elastic layers and the frangible layer, the elastic laminate may further include one or more facing layers that serve as an exterior surface of the laminate. The facing layer, for instance, may comprise a nonwoven material, such as a spunbond web or a meltblown web.

The lines of separation can be evenly spaced along the second direction of the laminate or can be unevenly spaced. In general, the density of the lines of separation can be from about one line of separation per about 50 mm to about one line of separation per 1 mm. In one embodiment, the density of the lines of separation is from about one line of separation per 10 mm to about one line of separation per 2 mm.

In an alternative embodiment, the present disclosure is directed to a laminate with elongation properties that includes a frangible layer positioned in between a first extensible layer and a second extensible layer. The first extensible layer may be elastic or non-elastic. Similarly, the second extensible layer may be elastic or non-elastic. In one embodiment, the laminate includes a frangible layer positioned between a first elastic layer and a second non-elastic extensible layer. The frangible layer can have the same elongation to break properties as described above in comparison to the first layer or in comparison to the second layer.

Of particular advantage, laminates with elongation properties can be made according to the present disclosure that have a relatively low basis weight, while having suitable physical properties for use in many commercial applications, such as use in absorbent articles. For instance, the basis weight of the laminate can be less than 110 gsm, such as from about 90 gsm to about 110 gsm. In other applications, however, it should be understood that the basis weight of the laminate can be greater than 110 gsm. The actual basis weight of the laminate may depend on various factors including the end use application. In other embodiments, for instance, the basis weight of the laminate can be greater than about 120 gsm, such as greater than about 150 gsm, such as even greater than about 200 gsm.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a cross-sectional view of one embodiment of an elastic laminate made in accordance with the present disclosure;

FIG. 2 is a plan view of another embodiment of a laminate made in accordance with the present disclosure;

FIG. 3 is a plan view of an alternative embodiment of an elastic laminate made in accordance with the present disclosure;

FIG. 4 is a perspective view of groove rolls that may be used in accordance with the present disclosure;

FIGS. 5-8 are one embodiment of an absorbent article made in accordance with the present disclosure;

FIG. 9 is a front view of another embodiment of an absorbent article made in accordance with the present disclosure; and

FIGS. 10-12 are cross sections of laminates made in accordance with the present disclosure. In FIG. 10, the frangible layer comprises an aluminum foil. In FIG. 11, the frangible layer comprises a meltblown layer. In FIG. 12, the frangible layer comprises a tissue web.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to laminates that have elongation properties, such as stretch properties. In one embodiment, the laminate may comprise an extensible laminate. The extensible laminate can also be elastic meaning that the laminate has stretch and recovery properties in at least one direction.

Laminates made according to the present disclosure include at least one extensible layer adhered to a frangible layer. The extensible layer can be non-elastic or elastic. In one embodiment, the frangible layer may be positioned in between a first extensible layer and a second extensible layer wherein each extensible layer can be elastic or non-elastic.

in one particular embodiment, the present disclosure is directed to an elastic laminate. The elastic laminate comprises at least one elastic layer adhered to a frangible layer.

In accordance with the present disclosure, the frangible layer includes lines of separation that extend in at least one direction. The lines of separation, for instance, can comprise lines where the frangible layer is weakened. In one embodiment, for instance, the frangible layer is severed into parallel strips. In alternative embodiments, the frangible layer is weakened such that the lines of separation have a lower basis weight than the remainder of the frangible layer. In still another embodiment, the lines of separation comprise areas where the frangible layer has been partially severed or cut. In still other embodiments, the lines of separation may comprise embossments that weaken the layer. In general, the lines of separation can comprise any lines of weakness that allow the frangible layer to separate when the laminate is elongated. In this manner, the frangible layer can be incorporated into the elastic laminate for providing at least one beneficial property without interfering with the ability of the extensible layer to be stretched in at least one direction. The frangible layer, for instance, can be incorporated into the laminate for increasing loft characteristics. The frangible layer can also serve as a liquid absorbent layer or even a conductive layer.

In one particular embodiment, the frangible layer is positioned in between a first extensible layer and a second extensible layer. After the frangible layer is positioned between the two extensible layers, the resulting laminate can be subjected to grooving which breaks the more rigid frangible layer without cutting or otherwise adversely impacting the other layers. Breaking the frangible layer allows the other layers to stretch freely. In one embodiment, the other layers can be bonded to one or more facing layers. The grooving of the laminate can be done either prior to attachment to the facing layers or after attachment to the facing layers depending upon the particular embodiment.

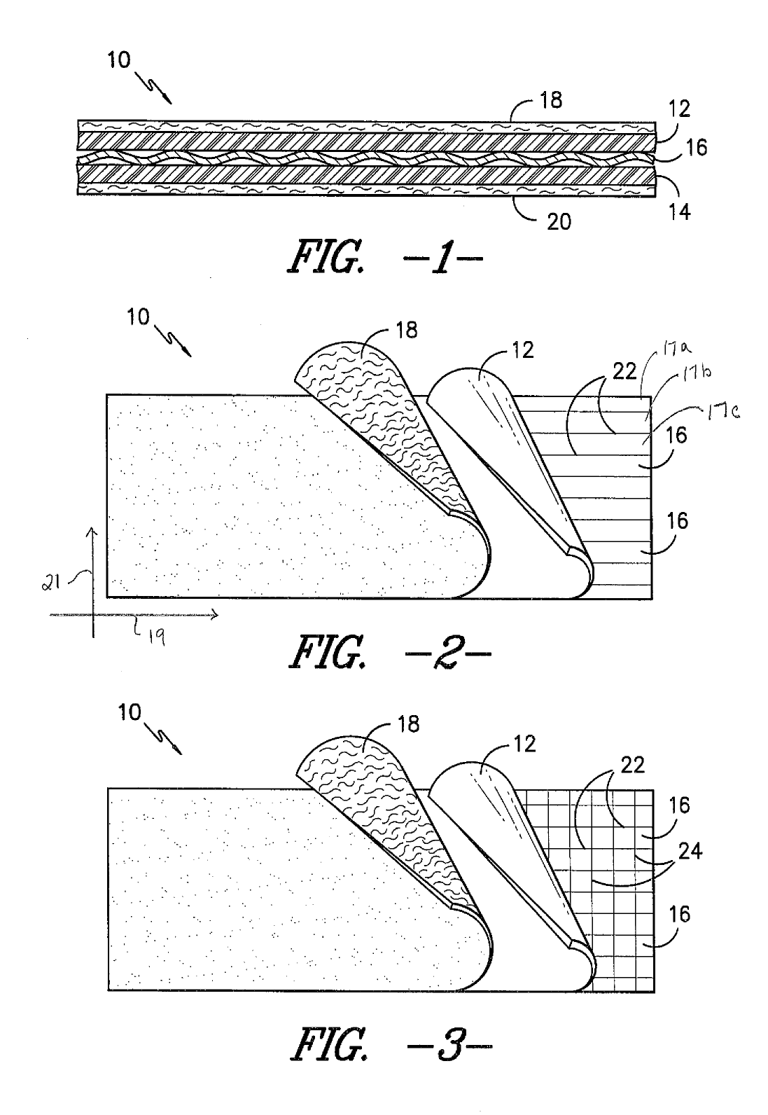

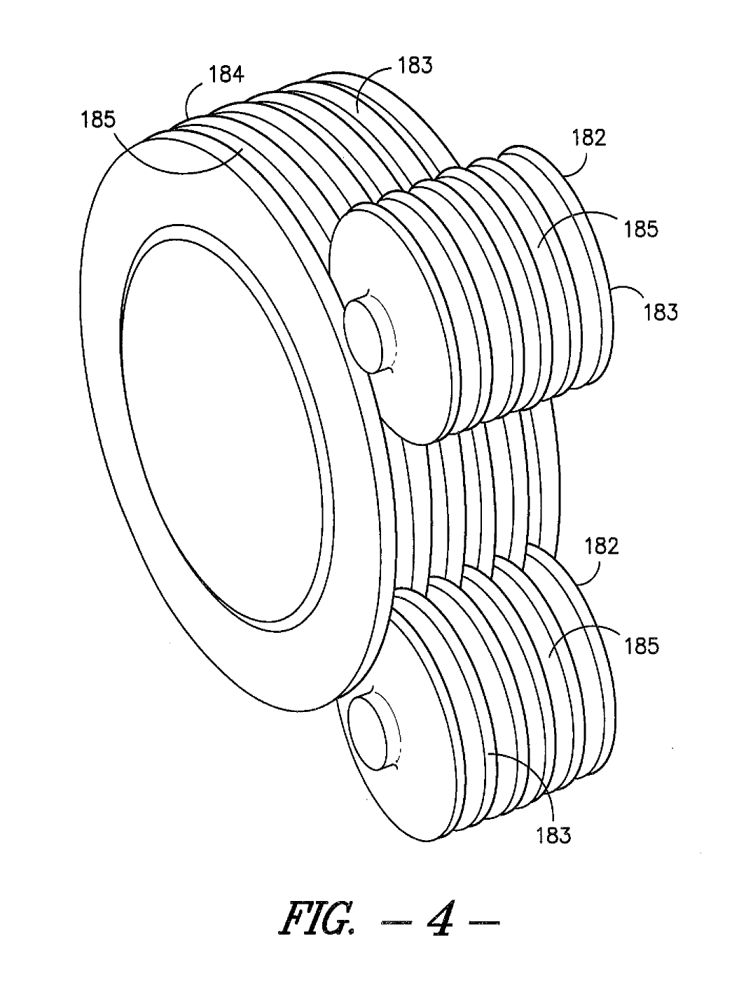

Referring to FIGS. 1 and 2, one embodiment of a laminate 10 made in accordance with the present disclosure is illustrated. As described above, laminates made in accordance with the present disclosure include at least one extensible layer and at least one frangible layer. The embodiment illustrated in FIG. 1 includes a first elastic layer 12 and a second elastic layer 14. Positioned in between the elastic layers 12 and 14 is a frangible layer 16. In the embodiment illustrated in FIG. 1, the elastic laminate 10 further includes a first facing layer 18 and a second facing layer 20. The first facing layer 18 is adjacent to and laminated to the first elastic layer 12, while the second facing layer 20 is adjacent to and laminated to the second elastic layer 14. The facing layers 18 and 20 can form opposing exterior surfaces of the laminate 10.

In one embodiment, the elastic layers 12 and 14 comprise elastic films, although the layers may also comprise elastic nonwoven materials. The frangible layer 16, on the other hand, comprises a layer that is more rigid and/or has lower elongation properties than the other layers and therefore is more easily severed when the laminate is subjected to severing and stretching processes such as groove rolling as is described subsequently herein.

For instance, referring to FIG. 2, the frangible layer 16 defines periodic lines of separation 22. The lines of separation 22 can, in one embodiment, form the frangible layer into a plurality of substantially parallel strips 17a, 17b, 17c, etc. As shown in FIG. 2, for instance, the lines of separation 22 generally extend in a first direction (shown by arrow 19). The lines of separation formed into the frangible layer 16 allow the elastic layers 12 and 14 to stretch freely in a direction (shown by arrow 21) perpendicular to the lines of separation 22. The lines of separation 22 not only allow the elastic layers to be stretched but also allow the elastic layers to recover after being stretched without any significant interference.

As shown in FIG. 2, the lines of separation 22 are generally parallel and are evenly spaced. In other embodiments, however, the lines of separation 22 may be unevenly spaced along a length or a width of the frangible layer 16.

The density of the lines of separation 22 can also vary depending upon the particular application. In one embodiment, for instance, the frangible layer 16 may include lines of separation that are spaced less than about 50 mm apart, such as less than about 40 mm apart, such as less than about 30 mm apart, such as less than about 20 mm apart, such as less than about 10 mm apart. For example, the lines of separation can be spaced less than 8 mm apart, such as less than about 6 mm apart, such as less than about 4 mm apart, such as even less than about 2 mm apart. In general, the lines of separation are spaced greater than about 0.5 mm apart, such as greater than about 1 mm apart. In one particular embodiment, the lines of separation can be spaced from about 2 mm apart to about 10 mm apart.

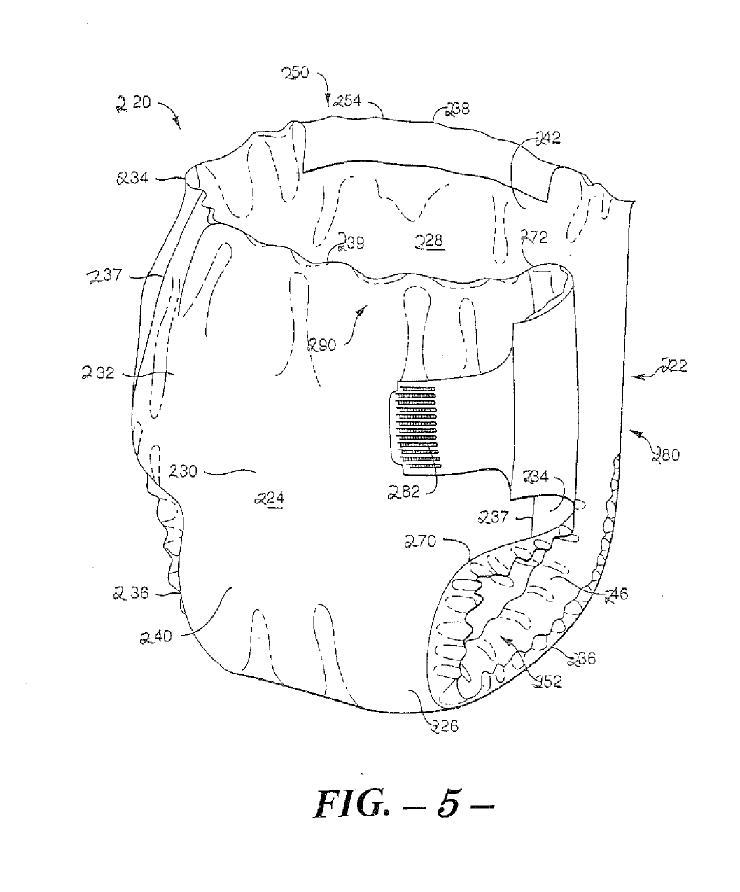

Referring to FIG. 3, another embodiment of an elastic laminate 10 made in accordance with the present disclosure is illustrated. Like reference numerals have been used to indicate similar elements. As shown in FIG. 3, the frangible layer 16 not only includes lines of separation 22 extending in a first direction 19 but also includes lines of separation 24 extending in a perpendicular direction 21. In this manner, the frangible layer 16 is formed into discrete shapes, such as rectangles or squares. Consequently, the elastic laminate can stretch and recover in multiple directions.

In general, the lines of separation can extend in the machine direction or in the cross-machine direction or both in the machine direction or in the cross-machine direction. In addition, the lines of separation can also extend in a diagonal direction to the machine direction. The lines of separation can be parallel or non-parallel and they can be straight or curved or combinations of the foregoing.

In the embodiment illustrated in FIGS. 1 and 2, layers 12 and 14 are indicated to be elastic layers. In other embodiments, however, layers 12 and 14 may comprise extensible but non-elastic layers. Extensible layers may comprise films and nonwoven materials.

The following is a more detailed description of the layers that may be used to produce the elastic laminate 10 as shown in the figures.

### Elastic Materials

In general, the elastic layer or elastomeric component can be an elastic film or an elastic nonwoven web. The elastomeric component can also be a single layer or a multi-layered material.

The elastic layers can be formed from one or more elastomeric polymers that are melt-processable, i.e. thermoplastic. In one embodiment, the elastic layer may be formed from a crosslinkable polymer. For instance, the elastic layer made from the polymer can be incorporated into the laminate and then subsequently crosslinked.

Any of a variety of thermoplastic elastomeric polymers may generally be employed in the present invention, such as elastomeric polyesters, elastomeric polyurethanes, elastomeric polyamides, elastomeric copolymers, elastomeric polyolefins, and so forth.

In general, any material known in the art to possess elastomeric characteristics can be used in the present invention as an elastomeric component. For example, suitable elastomeric resins include block copolymers having the general formula A-B-A′ or A-B, where A and A′ are each a thermoplastic polymer endblock which contains a styrenic moiety such as a polyvinyl arene) and where B is an elastomeric polymer midblock such as a conjugated diene or a lower alkene polymer. Block copolymers form the A and A′ blocks, and the present block copolymers are intended to embrace linear, branched and radial block copolymers.

In this regard, the radial block copolymers may be designated (A-B)ₘ-X, wherein X is a polyfunctional atom or molecule and in which each (A-B)ₘ-radiates from X in a way that A is an endblock. In the radial block copolymer, X may be an organic or inorganic polyfunctional atom or molecule and m is an integer having the same value as the functional group originally present in X. It is usually at least 3, and is frequently 4 or 5, but not limited thereto. Thus, in the present invention, the expression “block copolymer,” and particularly “A-B-A” and “A-B” block copolymer, is intended to embrace all block copolymers having such rubbery blocks and thermoplastic blocks as discussed above, which can be extruded (e.g., to make nonwovens or films), and without limitation as to the number of blocks. The elastomeric nonwoven web may be formed from, for example, elastomeric (polystyrene/poly(ethylene-butylene)/polystyrene) block copolymers. Commercial examples of such elastomeric copolymers are, for example, those known as KRATON materials which are available from Shell Chemical Company of Houston, Tex.

Polymers composed of an elastomeric A-B-A-B tetrablock copolymer may also be used in the practice of this invention. In such polymers, A is a thermoplastic polymer block and B is an isoprene monomer unit hydrogenated to substantially a poly(ethylene-propylene)monomer unit. An example of such a tetrablock copolymer is a styrene-poly(ethylene-propylene)-styrene-poly(ethylene-propylene) or SEPSEP elastomeric block copolymer available from the Shell Chemical Company of Houston, Tex. under the trade designation KRATON G-1657.

Other exemplary elastomeric materials which may be used include polyurethane elastomeric materials such as, for example, those available under the trademark ESTANE from B. F. Goodrich & Co. or MORTHANE from Morton Thiokol Corp., polyester elastomeric materials such as, for example, those available under the trade designation HYTREL from E. I. DuPont De Nemours & Company, and those known as ARNITEL, formerly available from Akzo Plastics of Amhem, Holland and now available from DSM of Sittard, Holland.

Another suitable material is a polyester block amide copolymer having the formula:

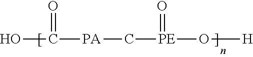

where n is a positive integer, PA represents a polyamide polymer segment and PE represents a polyether polymer segment.

Elastomeric polymers can also include copolymers of ethylene and at least one vinyl monomer such as, for example, vinyl acetates, unsaturated aliphatic monocarboxylic acids, and esters of such monocarboxylic acids. The elastomeric copolymers and formation of elastomeric nonwoven webs from those elastomeric copolymers are disclosed in, for example, U.S. Pat. No. 4,803,117.

The thermoplastic copolyester elastomers include copolyetheresters having the general formula:

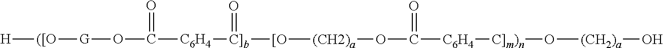

where “G” is selected from the group consisting of poly(oxyethylene)-alpha,omega-diol, poly(oxypropylene)-alpha,omega-diol, poly(oxytetramethylene)-alpha,omega-diol and “a” and “b” are positive integers including 2, 4 and 6, “m” and “n” are positive integers including 1-20.

Commercial examples of such copolyester materials are, for example, those known as ARNITEL, formerly available from Akzo Plastics of Amhem, Holland and now available from DSM of Sittard, Holland, or those known as HYTREL which are available from E. I. DuPont de Nemours of Wilmington, Del. Formation of an elastomeric nonwoven web from polyester elastomeric materials is disclosed in, for example, U.S. Pat. No. 4,741,949 to Morman et al. and U.S. Pat. No. 4,707,398 to Boggs which are herein incorporated by reference.

Elastomeric olefin polymers are available from Exxon Chemical Company of Baytown, Tex. under the trade name ACHIEVE for polypropylene based polymers and EXACT and EXCEED for polyethylene based polymers. Dow Chemical Company of Midland, Mich. has polymers commercially available under the name ENGAGE. These materials are believed to be produced using non-stereoselective metallocene catalysts. Exxon generally refers to their metallocene catalyst technology as “single site” catalysts while Dow refers to theirs as “constrained geometry” catalysts under the name INSIGHT to distinguish them from traditional Ziegler-Natta catalysts which have multiple reaction sites.

In other embodiments, the polymer used to produce the elastic layer may comprise a polypropylene plastomer or elastomer and/or a polyethylene plastomer or elastomer. Such polymers are sold under the name VISTAMAXX by Exxon Chemical Company. Another class of suitable polymers that may be used include propylene-ethylene copolymers sold under the name VERSIFY by the Dow Chemical Company. Such polymers may comprise infused olefinic block copolymers.

Fibrous elastic webs can also be formed from an extruded polymer. For instance, as stated above, in one embodiment the fibrous web can contain meltblown fibers. The fibers can be continuous or discontinuous. Meltblown fabrics have been conventionally made by extruding a thermoplastic polymeric material through a die to form fibers. As the molten polymer fibers exit the die, a high pressure fluid, such as heated air or steam, attenuates the molten polymer filaments to form fine fibers. Surrounding cool air is induced into the hot air stream to cool and solidify the fibers. The fibers are then randomly deposited onto a foraminous surface to form a web. The web has integrity but may be additionally bonded if desired.

Besides meltblown webs, however, it should be understood that other fibrous webs can be used in accordance with the present invention. For instance, in an alternative embodiment, elastic spunbond webs can also be formed from spunbond fibers. Spunbond webs are typically produced by heating a thermoplastic polymeric resin to at least its softening temperature, then extruding it through a spinnerette to form continuous fibers, which can be subsequently fed through a fiber draw unit. From the fiber draw unit, the fibers are spread onto a foraminous surface where they are formed into a web and then bonded such as by chemical, thermal or ultrasonic means.

The amount of elastomeric polymer(s) employed in the elastic layer may vary, but is typically about 30 wt. % or more of the layer, in some embodiments about 50 wt. % or more, and in some embodiments, about 80 wt. % or more of the layer.

Besides polymers, the elastic layer may also contain other components as is known in the art. In one embodiment, for example, the elastic layer contains a filler. Fillers are particulates or other forms of material that may be added to the film polymer extrusion blend and that will not chemically interfere with the elastic layer, but which may be uniformly dispersed throughout the layer. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways.

The fillers may have a spherical or non-spherical shape with average particle sizes in the range of from about 0.1 to about 7 microns. Examples of suitable fillers include, but are not limited to, calcium carbonate, various kinds of clay, silica, alumina, barium carbonate, sodium carbonate, magnesium carbonate, talc, barium sulfate, magnesium sulfate, aluminum sulfate, titanium dioxide, zeolites, cellulose-type powders, kaolin, mica, carbon, calcium oxide, magnesium oxide, aluminum hydroxide, pulp powder, wood powder, cellulose derivatives, chitin and chitin derivatives. A suitable coating, such as stearic acid, may also be applied to the filler particles if desired. When utilized, the filler content may vary, such as from about 25 wt. % to about 75 wt. %, in some embodiments, from about 30 wt. % to about 70 wt. %, and in some embodiments, from about 40 wt. % to about 60 wt. % of the elastic layer.

Other additives may also be incorporated into the elastic layer, such as melt stabilizers, processing stabilizers, heat stabilizers, light stabilizers, antioxidants, heat aging stabilizers, whitening agents, antiblocking agents, bonding agents, tackifiers, viscosity modifiers, etc. Examples of suitable tackifier resins may include, for instance, hydrogenated hydrocarbon resins. REGALREZ™ hydrocarbon resins are examples of such hydrogenated hydrocarbon resins, and are available from Eastman Chemical. Other tackifiers are available from ExxonMobil under the ESCOREZ™ designation. Viscosity modifiers may also be employed, such as polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox® 1076, 1010, or E 201. Moreover, bonding agents may also be added to the film to facilitate bonding of the film to additional materials (e.g., nonwoven web). When employed, such additives (e.g., tackifier, antioxidant, stabilizer, etc.) may each be present in an amount from about 0.001 wt. % to about 25 wt. %, in some embodiments, from about 0.005 wt. % to about 20 wt. %, and in some embodiments, from 0.01 wt. % to about 15 wt. % of the elastic layer.

The elastic layer may be mono- or multi-layered. Multilayer films may be prepared by co-extrusion of the layers, extrusion coating, or by any conventional layering process. Such multilayer films normally contain at least one base layer and at least one skin layer, but may contain any number of layers desired. For example, the multilayer film may be formed from a base layer and one or more skin layers, wherein the base layer is formed from an elastomeric polymer. In such embodiments, the skin layer(s) may be formed from any film-forming polymer. If desired, the skin layer(s) may contain a softer, lower melting polymer or polymer blend that renders the layer(s) more suitable as heat seal bonding layers for thermally bonding the film to other layers. For example, the skin layer(s) may be formed from an olefin polymer or blends thereof. Additional film-forming polymers that may be suitable for use with the present invention, alone or in combination with other polymers, include ethylene vinyl acetate, ethylene ethyl acrylate, ethylene acrylic acid, ethylene methyl acrylate, ethylene normal butyl acrylate, nylon, ethylene vinyl alcohol, polystyrene, polyurethane, and so forth.

The thickness of the skin layer(s) is generally selected so as not to substantially impair the elastomeric properties of the film. To this end, each skin layer may separately comprise from about 0.5% to about 15% of the total thickness of the film, and in some embodiments from about 1% to about 10% of the total thickness of the film. For instance, each skin layer may have a thickness of from about 0.1 to about 10 micrometers, in some embodiments from about 0.5 to about 5 micrometers, and in some embodiments, from about 1 to about 2.5 micrometers. Likewise, the base layer may have a thickness of from about 1 to about 40 micrometers, in some embodiments from about 2 to about 25 micrometers, and in some embodiments, from about 5 to about 20 micrometers.

The properties of the resulting film may generally vary as desired. For instance, prior to stretching, the film typically has a basis weight of about 100 grams per square meter or less, and in some embodiments, from about 20 to about 75 grams per square meter.

### Extensible and Non-Elastic Layer

In one embodiment, one or more layers of the laminate may be extensible but non-elastic. Such layers may comprise films or nonwoven webs. Various nonwoven webs including meltblown webs, spunbond webs, bonded carded webs and the like can have extensible properties in at least one direction.

Extensible but non-elastic films can be made from various different polymers. Such polymers include low density polyethylene polymers, such as LDPE 113C available from the Dow Chemical Company. In another embodiment, a polyethylene resin, such as DOWLEX 2028B can be used to produce the film. In still another embodiment, the extensible film can be made from a polypropylene polymer, such as a polypropylene copolymer. One suitable polypropylene copolymer is sold under the name ACCUCOMP CP0400L by ACLO Compounders, Inc.

### Frangible Layer

In general, the frangible layer can comprise any suitable material that is more rigid and stiff than the elastic layers, has a higher elastic modulus, has a lower elongation to break, and/or that provides some benefit to the elastic laminate based upon the end use application. The frangible layer may comprise a nonwoven material, such as a paper web including tissue webs. The frangible layer may also comprise a meltspun web including a meltblown layer, a spunbond layer, a hydroentangled material, a bonded carded material, or a coform material. In still other embodiments, the frangible layer may comprise a film or a foil. In one embodiment, the frangible layer may comprise a metal foil, such as aluminum foil, that can make the elastic laminate conductive in at least one direction.

In one particular embodiment, the frangible layer comprises a tissue web. A tissue web can substantially increase the loft characteristics of the elastic laminate. Tissue webs are also water absorbent.

The basis weight of the frangible layer when a nonwoven is used may generally vary, such as from about 5 grams per square meter (“gsm”) to 120 gsm, in some embodiments from about 10 gsm to about 70 gsm, and in some embodiments, from about 15 gsm to about 35 gsm. Lower basis weight nonwoven materials may be preferred in some applications. For instance, lower basis weight materials may allow for the overall basis weight of the laminate to be relatively low, such as less than about 110 gsm. In other embodiments, however, higher basis weight laminates may be produced from nonwoven materials having basis weights well above 35 gsm.

Fibers suitable for making tissue webs comprise any natural or synthetic cellulosic fibers including, but not limited to nonwoody fibers, such as cotton, abaca, kenaf, sabai grass, flax, esparto grass, straw, jute hemp, bagasse, milkweed floss fibers, bamboo fibers, algae fibers, corn stover fibers, and pineapple leaf fibers; and woody or pulp fibers such as those obtained from deciduous and coniferous trees, including softwood fibers, such as northern and southern softwood kraft fibers; hardwood fibers, such as eucalyptus, maple, birch, and aspen. Pulp fibers can be prepared in high-yield or low-yield forms and can be pulped in any known method, including kraft, sulfite, high-yield pulping methods and other known pulping methods.

A portion of the fibers, such as up to 50% or less by dry weight, or from about 5% to about 30% by dry weight, can be synthetic fibers such as rayon, polyolefin fibers, polyamide fibers, polyester fibers, bicomponent sheath-core fibers, multi-component binder fibers, and the like. An exemplary polyethylene fiber is Fybrel®, available from Minifibers, Inc. (Jackson City, Tenn.). Any known bleaching method can be used. Synthetic cellulose fiber types include rayon in all its varieties and other fibers derived from viscose or chemically-modified cellulose. Chemically treated natural cellulosic fibers can be used such as mercerized pulps, chemically stiffened or crosslinked fibers, or sulfonated fibers. For good mechanical properties in using papermaking fibers, it can be desirable that the fibers be relatively undamaged and largely unrefined or only lightly refined. While recycled fibers can be used, virgin fibers are generally useful for their mechanical properties and lack of contaminants. Mercerized fibers, regenerated cellulosic fibers, cellulose produced by microbes, rayon, and other cellulosic material or cellulosic derivatives can be used. Suitable papermaking fibers can also include recycled fibers, virgin fibers, or mixes thereof. In certain embodiments capable of high bulk and good compressive properties, the fibers can have a Canadian Standard Freeness of at least 200, more specifically at least 300, more specifically still at least 400, and most specifically at least 500.

Other papermaking fibers that can be used in the present disclosure include paper broke or recycled fibers and high yield fibers. High yield pulp fibers are those papermaking fibers produced by pulping processes providing a yield of about 65% or greater, more specifically about 75% or greater, and still more specifically about 75% to about 95%. Yield is the resulting amount of processed fibers expressed as a percentage of the initial wood mass. Such pulping processes include bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), high yield sulfite pulps, and high yield Kraft pulps, all of which leave the resulting fibers with high levels of lignin. High yield fibers are well known for their stiffness in both dry and wet states relative to typical chemically pulped fibers.

In general, any process capable of forming a sheet can also be utilized in the present disclosure to form the frangible layer. For example, a papermaking process of the present disclosure can utilize creping, wet creping, double creping, embossing, wet pressing, air pressing, through-air drying, creped through-air drying, uncreped through-air drying, hydroentangling, air laying, coform methods, as well as other steps known in the art.

Also suitable for use as the frangible layer of the present disclosure are tissue sheets that are pattern densified or imprinted, such as the tissue sheets disclosed in any of the following U.S. Pat. No. 4,514,345 issued on Apr. 30, 1985, to Johnson et al.; U.S. Pat. No. 4,528,239 issued on Jul. 9, 1985, to Trokhan; U.S. Pat. No. 5,098,522 issued on Mar. 24, 1992; U.S. Pat. No. 5,260,171 issued on Nov. 9, 1993, to Smurkoski et al.; U.S. Pat. No. 5,275,700 issued on Jan. 4, 1994, to Trokhan; U.S. Pat. No. 5,328,565 issued on Jul. 12, 1994, to Rasch et al.; U.S. Pat. No. 5,334,289 issued on Aug. 2, 1994, to Trokhan et al.; U.S. Pat. No. 5,431,786 issued on Jul. 11, 1995, to Rasch et al.; U.S. Pat. No. 5,496,624 issued on Mar. 5, 1996, to Steltjes, Jr. et al.; U.S. Pat. No. 5,500,277 issued on Mar. 19, 1996, to Trokhan et al.; U.S. Pat. No. 5,514,523 issued on May 7, 1996, to Trokhan et al.; U.S. Pat. No. 5,554,467 issued on Sep. 10, 1996, to Trokhan et al.; U.S. Pat. No. 5,566,724 issued on Oct. 22, 1996, to Trokhan et al.; U.S. Pat. No. 5,624,790 issued on Apr. 29, 1997, to Trokhan et al.; and, U.S. Pat. No. 5,628,876 issued on May 13, 1997, to Ayers et al., the disclosures of which are incorporated herein by reference to the extent that they are non-contradictory herewith. Such imprinted tissue sheets may have a network of densified regions that have been imprinted against a drum dryer by an imprinting fabric, and regions that are relatively less densified (e.g., “domes” in the tissue sheet) corresponding to deflection conduits in the imprinting fabric, wherein the tissue sheet superposed over the deflection conduits was deflected by an air pressure differential across the deflection conduit to form a lower-density pillow-like region or dome in the tissue sheet.

The tissue web can also be formed without a substantial amount of inner fiber-to-fiber bond strength. In this regard, the fiber furnish used to form the base web can be treated with a chemical debonding agent. The debonding agent can be added to the fiber slurry during the pulping process or can be added directly to the headbox. Suitable debonding agents that may be used in the present disclosure include cationic debonding agents such as fatty dialkyl quaternary amine salts, mono fatty alkyl tertiary amine salts, primary amine salts, imidazoline quaternary salts, silicone quaternary salt and unsaturated fatty alkyl amine salts. Other suitable debonding agents are disclosed in U.S. Pat. No. 5,529,665 to Kaun which is incorporated herein by reference. In particular, Kaun discloses the use of cationic silicone compositions as debonding agents.

In one embodiment, the debonding agent used in the process of the present disclosure is an organic quaternary ammonium chloride and, particularly, a silicone-based amine salt of a quaternary ammonium chloride. For example, the debonding agent can be PROSOFT® TQ1003, marketed by the Hercules Corporation. The debonding agent can be added to the fiber slurry in an amount of from about 1 kg per metric tonne to about 10 kg per metric tonne of fibers present within the slurry.

In an alternative embodiment, the debonding agent can be an imidazoline-based agent. The imidazoline-based debonding agent can be obtained, for instance, from the Witco Corporation. The imidazoline-based debonding agent can be added in an amount of between 2.0 to about 15 kg per metric tonne.

Optional chemical additives may also be added to the aqueous papermaking furnish or to the formed embryonic web to impart additional benefits to the product and process and are not antagonistic to the intended benefits of the invention. The following materials are included as examples of additional chemicals that may be applied to the web along with the additive composition of the present invention. The chemicals are included as examples and are not intended to limit the scope of the invention. Such chemicals may be added at any point in the papermaking process, including being added simultaneously with the additive composition in the pulp making process, wherein said additive or additives are blended directly with the additive composition.

Additional types of chemicals that may be added to the paper web include, but is not limited to, absorbency aids usually in the form of cationic, anionic, or non-ionic surfactants, humectants and plasticizers such as low molecular weight polyethylene glycols and polyhydroxy compounds such as glycerin and propylene glycol. Materials that supply skin health benefits such as mineral oil, aloe extract, vitamin e, silicone, lotions in general and the like may also be incorporated into the finished products.

In general, the products of the present invention can be used in conjunction with any known materials and chemicals that are not antagonistic to its intended use. Examples of such materials include but are not limited to odor control agents, such as odor absorbents, activated carbon fibers and particles, baby powder, baking soda, chelating agents, zeolites, perfumes or other odor-masking agents, cyclodextrin compounds, oxidizers, and the like. Superabsorbent particles, synthetic fibers, or films may also be employed. Additional options include cationic dyes, optical brighteners, humectants, emollients, and the like.

The different chemicals and ingredients that may be incorporated into the frangible layer may depend upon the end use of the product. For instance, various wet strength agents may be incorporated into the product. For tissue webs, for example, temporary wet strength agents may be used. As used herein, wet strength agents are materials used to immobilize the bonds between fibers in the wet state. Typically, the means by which fibers are held together in paper and tissue products involve hydrogen bonds and sometimes combinations of hydrogen bonds and covalent and/or ionic bonds. In some applications, it may be useful to provide a material that will allow bonding to the fibers in such a way as to immobilize the fiber-to-fiber bond points and make them resistant to disruption in the wet state. The wet state typically means when the product is largely saturated with water or other aqueous solutions.

Any material that when added to a paper or tissue web results in providing the sheet with a mean wet geometric tensile strength:dry geometric tensile strength ratio in excess of 0.1 may be termed a wet strength agent.

Temporary wet strength agents, which are typically incorporated into bath tissues, are defined as those resins which, when incorporated into paper or tissue products, will provide a product which retains less than 50% of its original wet strength after exposure to water for a period of at least 5 minutes. Temporary wet strength agents are well known in the art. Examples of temporary wet strength agents include polymeric aldehyde-functional compounds such as glyoxylated polyacrylamide, such as a cationic glyoxylated polyacrylamide.

Such compounds include PAREZ 631 NC wet strength resin available from Cytec Industries of West Patterson, N.J., chloroxylated polyacrylamides, and HERCOBOND 1366, manufactured by Hercules, Inc. of Wilmington, Del. Another example of a glyoxylated polyacrylamide is PAREZ 745, which is a glyoxylated poly(acrylamide-co-diallyl dimethyl ammonium chloride).

Permanent wet strength agents may also be incorporated into the base sheet. Permanent wet strength agents are also well known in the art and provide a product that will retain more than 50% of its original wet strength after exposure to water for a period of at least 5 minutes.

Tissue webs may include a single homogenous layer of fibers or may include a stratified or layered construction. For instance, the tissue web ply may include two or three layers of fibers. Each layer may have a different fiber composition.

Forming multi-layered paper webs is described and disclosed in U.S. Pat. No. 5,129,988 to Farrington, Jr., which is incorporated herein by reference.

The basis weight of tissue webs used in accordance with the present disclosure can vary depending upon the final product. In general, the basis weight of the tissue web may vary from about 10 gsm to about 110 gsm, such as from about 15 gsm to about 40 gsm.

The tissue web bulk may also vary from about 3 cc/g to 20 cc/g, such as from about 5 cc/g to 15 cc/g. The sheet “bulk” is calculated as the quotient of the caliper of a dry tissue sheet, expressed in microns, divided by the dry basis weight, expressed in grams per square meter. The resulting sheet bulk is expressed in cubic centimeters per gram. More specifically, the caliper is measured as the total thickness of a stack of ten representative sheets and dividing the total thickness of the stack by ten, where each sheet within the stack is placed with the same side up. Caliper is measured in accordance with TAPPI test method T411 om-89 “Thickness (caliper) of Paper, Paperboard, and Combined Board” with Note 3 for stacked sheets. The micrometer used for carrying out T411 om-89 is an Emveco 200-A Tissue Caliper Tester available from Emveco, Inc., Newberg, Oreg. The micrometer has a load of 2.00 kilo-Pascals (132 grams per square inch), a pressure foot area of 2500 square millimeters, a pressure foot diameter of 56.42 millimeters, a dwell time of 3 seconds and a lowering rate of 0.8 millimeters per second.

In one embodiment, the tissue web is formed by an uncreped through-air drying process. In this embodiment, a wet web is dried on a through-air dryer without being creped. Tissue webs made according to the above process can be produced that have a significant amount of surface texture which may increase the bulk of laminates incorporating the web.

In one embodiment, for instance, the web may be transferred to a throughdrying fabric for final drying preferably with the assistance of vacuum to ensure macroscopic rearrangement of the web to give the desired bulk and appearance. The throughdrying fabrics are designed to deliver bulk and stretch. It is therefore useful to have throughdrying fabrics which are quite coarse and three dimensional in one embodiment. The result is that a sheet is macroscopically rearranged (with vacuum assist) to give the high bulk, high stretch surface topology of the throughdrying fabric. Sheet topology is completely changed from transfer to throughdrying fabric and fibers are macroscopically rearranged, including significant fiber-fiber movement.

The drying process can be any noncompressive drying method which tends to preserve the bulk or thickness of the wet web including, without limitation, throughdrying, infra-red radiation, microwave drying, etc.

The topographical surface used to mold the tissue web generally comprises a porous material containing elevations that extend from the surface. Many different types of materials may be used as the topographical surface. In one particular embodiment, for instance, the topographical surface comprises a three dimensional papermaking fabric, such as those disclosed in U.S. Pat. No. 8,105,463 which is incorporated herein by reference.

A woven papermaking fabric, which has a topography that can form ridges and valleys in the tissue sheet when the dewatered sheet is molded to conform to its surface. More particularly, a texturizing fabric is a woven papermaking fabric having a textured sheet contacting surface with substantially continuous machine-direction elevations or ripples separated by valleys, the ripples being formed of multiple warp strands grouped together and supported by multiple shute strands of one or more diameters; wherein the width of ripples is from about 1 to about 5 millimeters, more specifically from about 1.3 to about 3 millimeters, and still more specifically from about 1.9 to about 2.4 millimeters. The frequency of occurrence of the ripples in the cross-machine direction of the fabric is from about 0.5 to about 8 per centimeter, more specifically from about 3.2 to about 7.9, still more specifically from about 4.2 to about 5.3 per centimeter. The rippled channel depth, which is the z-directional distance between the top plane of the fabric and the lowest visible fabric knuckle that the tissue web may contact, can be from about 0.2 to about 1.6 millimeters, more specifically from about 0.7 to about 1.1 millimeters, and still more specifically from about 0.8 to about 1 millimeter. For purposes herein, a “knuckle” is a structure formed by overlapping warp and shute strands.

It should be understood, however, the use of a three-dimensional fabric merely represents one embodiment of a topographical surface used in the process. For instance, in other embodiments discrete shapes such as deflection elements may be mounted on a porous substrate for forming the elevations.

In addition to uncreped through-air dried webs, various other tissue webs may be used in the laminate. For example, in an alternative embodiment, the tissue web may comprise a wet pressed web. A web pressed web is a web that is transferred to the surface of a rotatable heated dryer drum, such as a Yankee dryer. In one embodiment, a creping adhesive may be applied to the web or to the drum. While on the surface of the rotating drum, the web is dried and then creped from the surface.

In still another embodiment, the tissue web may comprise a print-crepe web. During a print-crepe process, a formed tissue web, such as an uncreped through-air dried web, is treated with an adhesive or bonding material. The adhesive or bonding material may be applied to the web in a pattern. The adhesive or bonding material is used to adhere the web to a creping drum. The web is then creped from the drum.

In one embodiment, only one side of the tissue web may be fed through a print-crepe process. In an alternative embodiment, both sides of the web can be printed with an adhesive or bonding material and creped.

The bonding material is applied to each side of the paper web so as to cover from about 15% to about 75% of the surface area of the web. More particularly, in most applications, the bonding material will cover from about 20% to about 60% of the surface area of each side of the web. The total amount of bonding material applied to each side of the web can be in the range of from about 1% to about 30% by weight, based upon the total weight of the web, such as from about 1% to about 20% by weight, such as from about 2% to about 10% by weight.

At the above amounts, the bonding material can penetrate the tissue web after being applied in an amount up to about 30% of the total thickness of the web, depending upon various factors. It has been discovered, however, that most of the bonding material primarily resides on the surface of the web after being applied to the web. For instance, in some embodiments, the bonding material penetrates the web less than 5%, such as less than 3%, such as less than 1% of the thickness of the web.

The bonding material applied to the tissue web can vary depending upon the particular application. In general, the bonding material may comprise an ethylene vinyl acetate copolymer. In an alternative embodiment, the bonding material may comprise a polyolefin polymer. For instance, in one embodiment, an α-olefin interpolymer may be applied to the tissue web as an aqueous dispersion. In this embodiment, the aqueous dispersion may contain a dispersing agent such as an ethylene acrylic acid copolymer.

In addition to paper webs such as tissue webs, the frangible layer may also comprise various other nonwoven materials. For instance, in other embodiments, the frangible layer may comprise a meltspun web, such as a spunbond web or a meltblown web. The meltspun web can be made from polymer materials that are relatively rigid and stiff and that will break and separate when subjected to a groove roll. When using a spunbond or meltblown web, the basis weight of the material may be relatively low. For instance, the basis weight may be less than about 20 gsm, such as less than about 15 gsm, such as less than about 10 gsm, such as even less than about 5 gsm.

In still another embodiment, the frangible layer may comprise a nonwoven material that contains synthetic fibers and pulp fibers. For instance, the frangible layer may also comprise a hydroentangled web or a coform web.

Hydroentangled webs, which are also known as spunlace webs, refer to webs that have been subjected to columnar jets of a fluid that cause the fibers in the web to entangle. For example, in one embodiment, the base web can comprise HYDROKNIT7, a nonwoven composite fabric that contains 70% by weight pulp fibers that are hydraulically entangled into a continuous filament material. HYDROKNIT7 material is commercially available from Kimberly-Clark Corporation of Neenah, Wis. Hydraulic entangling may be accomplished utilizing conventional hydraulic entangling equipment such as may be found in, for example, in U.S. Pat. No. 3,485,706 to Evans or U.S. Pat. No. 5,389,202 to Everhart et al., the disclosures of which are hereby incorporated by reference.

Still another example of suitable materials for the frangible layer includes coform materials. In general, “coform” means a process in which at least one meltblown in die is arranged near a chute through which other materials are added to the web while it forms. Such other materials can include, for example, pulp, superabsorbent particles, or cellulose or staple fibers or mixtures thereof. Coform processes are described in U.S. Pat. No. 4,818,464 to Lau and U.S. Pat. No. 4,100,324 to Anderson et al., which are incorporated by reference. Webs produced by the coform process are generally referred to as coform materials.

In still another embodiment, the frangible layer may comprise a metal layer, such as a metal foil. For instance, the metal foil may comprise aluminum foil.

### Lamination

In order to combine the frangible layer with one or more extensible layers, various techniques and methods can be used to attach the different materials together without limitation. In one embodiment, for instance, the materials can be attached together through thermal bonding or ultrasonic bonding. In one embodiment, the materials can be bonded together according to a pattern. In this embodiment, the materials can be point bonded together using either thermal bonding or ultrasonic bonding. Bonding can occur before, during or after the lines of separation are formed.

In an alternative embodiment, the materials can be bonded together using an adhesive.

The materials can be attached together either in a relaxed state or in a stretched state. In one embodiment, for instance, one of the extensible layers can be in a stretched state when attached to the frangible layer or to another layer in the laminate. In one embodiment, one of the layers can also be a necked layer when the laminate is constructed. In a necked state, a nonwoven web is stretched so as to have a necked width that is less than the starting width of the material. In another embodiment, the layers incorporated into the laminate can be attached together when all of the layers are in a non-stretched state.

The resulting laminate can be liquid impervious, especially when the extensible layers comprise films. In one embodiment, the laminate can be liquid impervious but yet remain breathable. In still another embodiment, the laminate may be liquid pervious.

In one embodiment, at least one of the extensible layers may comprise an apertured film. The apertured film may be breathable but liquid impermeable. In an alternative embodiment, the apertured film may be liquid permeable. In one embodiment, the apertured film may be liquid permeable so that the frangible layer may absorb liquids. In this embodiment, the frangible layer may contain absorbent particles, such as superabsorbent particles.

### Forming Lines of Separation

In one embodiment, once the laminate is produced, lines of separation can be formed into the frangible layer. In one embodiment, for instance, the laminate may be fed between a first roller and a second roller wherein at least one of the rollers defines grooves. The laminate is fed in between the two rollers with sufficient nip pressure to form the lines of separation in the frangible layer without cutting the first extensible layer or the second extensible layer. In one embodiment, the groove roll may also bond the laminate together as the lines of separation are formed.

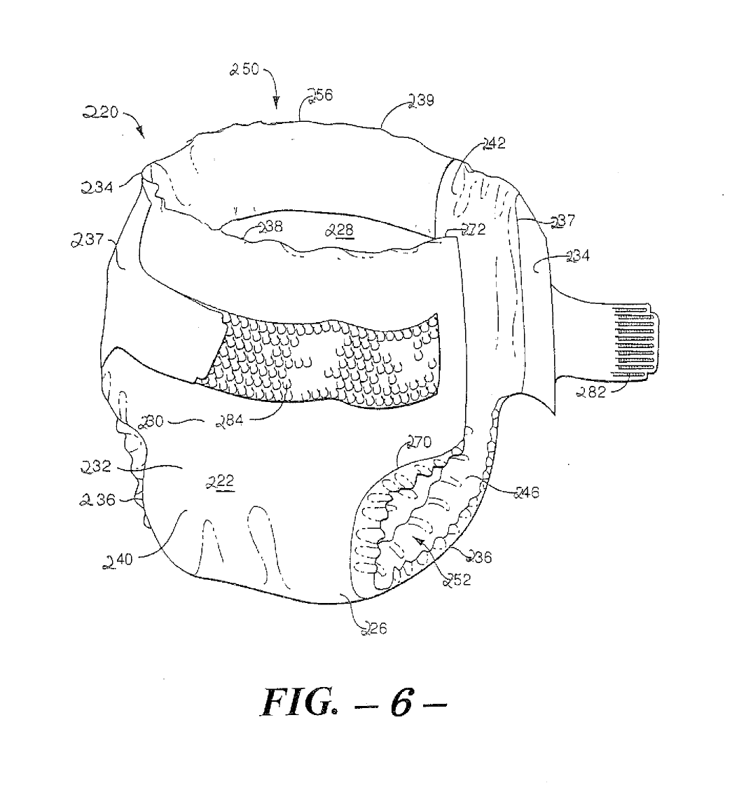

Referring to FIG. 4, one embodiment of groove rolls that may be used in accordance with the present disclosure is illustrated. As shown, for example, satellite rolls 182 may engage an anvil roll 184, each of which include a plurality of ridges 183 defining a plurality of grooves 185 positioned across the grooved rolls in the cross-machine direction. The grooves 185 are generally oriented perpendicular to the direction of stretch of the material. In other words, the grooves 185 may be oriented in the machine direction. The grooves 185 may likewise be oriented in the cross-machine direction. The ridges 183 of satellite roll 182 intermesh with the grooves 185 of anvil roll 184, and the grooves 185 of satellite roll 182 intermesh with the ridges 183 of anvil roll 184.

The dimensions and parameters of the grooves 185 and ridges 183 may vary. For example, the number of grooves 185 contained on a roll may vary depending on the number of lines of separation to be formed. The grooves 185 may also have a certain depth “D”, which generally ranges from about 2 mm to about 20 mm, and in some embodiments, from about 8 mm to about 15 mm. In addition, the peak-to-peak distance “P” between the grooves 185 is typically from about 1 mm to about 50 mm, and in some embodiments, from about 2 mm to about 10 mm.

In general, the groove rolls can include grooves that are evenly spaced along the length of the groove face or unevenly spaced. In various embodiments, the density of grooves can be from about 1 groove per about 50 mm to about 1 groove per about 1 mm. In other embodiments, the grooves can be spaced such that there is 1 groove per about 2 mm to about 1 groove per about 7 mm.

If desired, heat may be applied to the composite or laminate just prior to or during the application of the grooves to cause it to relax somewhat and ease extension. Heat may be applied by any suitable method known in the art, such as heated air, infrared heaters, heated nipped rolls, or partial wrapping of the laminate around one or more heated rolls or steam canisters, etc. Heat may also be applied to the grooved rolls themselves. It should also be understood that other grooved roll arrangement are equally suitable, such as two grooved rolls positioned immediately adjacent to one another. In another embodiment, the process may include a grooved roll that contacts a flat anvil roll which may have a deformable surface.

### Embodiments

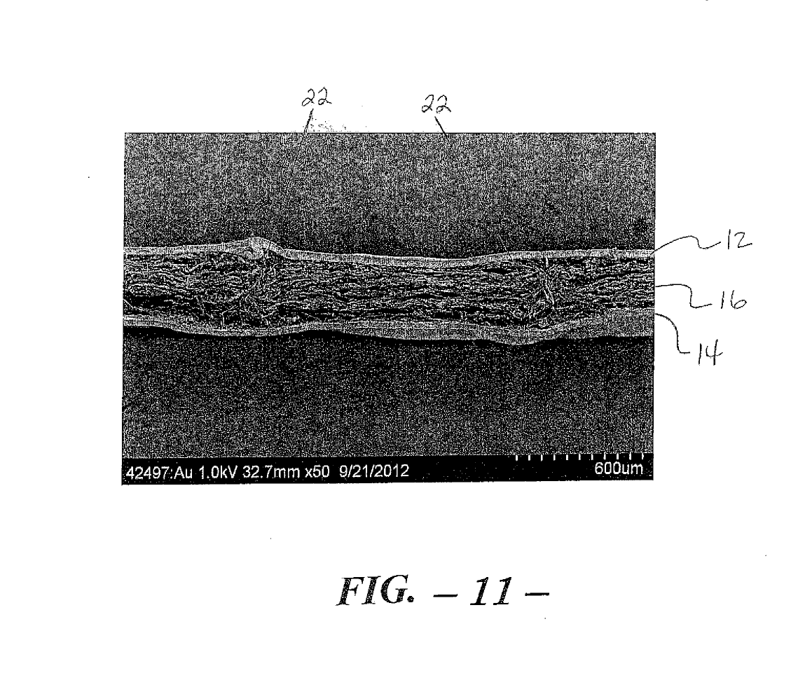

Embodiments of laminates made in accordance with the present disclosure are shown in FIGS. 10-12. In each of the embodiments, a frangible layer was placed in between two elastic layers. Each elastic layer comprised a styrenic polymer, particularly an SIBS polymer. Each elastic layer had a skin layer comprised of a blend of linear low density polyethylenes.

In FIG. 10, the laminate includes a first elastic layer 12, a second elastic layer 14, and a frangible layer 16. The frangible layer comprises an aluminum foil and includes lines of separation 22.

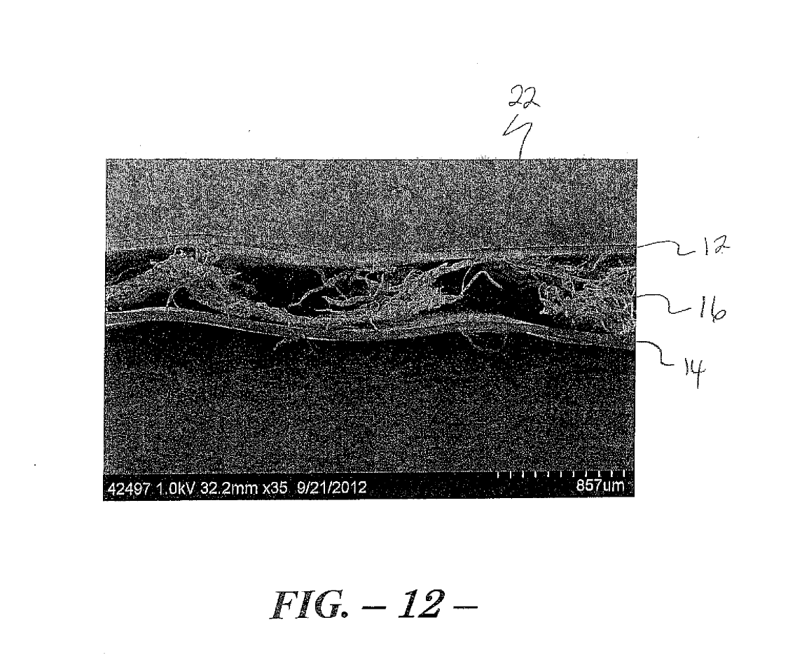

In FIG. 11, the frangible layer 16 comprises a meltblown web made from polypropylene fibers and having a basis weight of 26 gsm. The lines of separation 22 show weakened areas of the web 16.

In the embodiment illustrated in FIG. 12, the frangible layer 16 comprises a tissue web having a basis weight of 15 gsm. As shown, the lines of separation 22 are areas where the tissue web 16 has been weakened. A portion of the thickness of the web has been severed. Consequently, the lines of weakness 22 are areas where the basis weight of the web is less than the basis weight of the remainder of the web.

### Applications

The laminate of the present invention may be used in a wide variety of applications. For example, the laminate may be used in an absorbent article. An “absorbent article” generally refers to any article capable of absorbing water or other fluids. Examples of some absorbent articles include, but are not limited to, Personal care absorbent articles, such as diapers, diaper pants, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Absorbent articles may include a substantially liquid-impermeable layer (e.g., outer cover), a liquid-permeable layer (e.g., bodyside liner, surge layer, etc.), and an absorbent core.

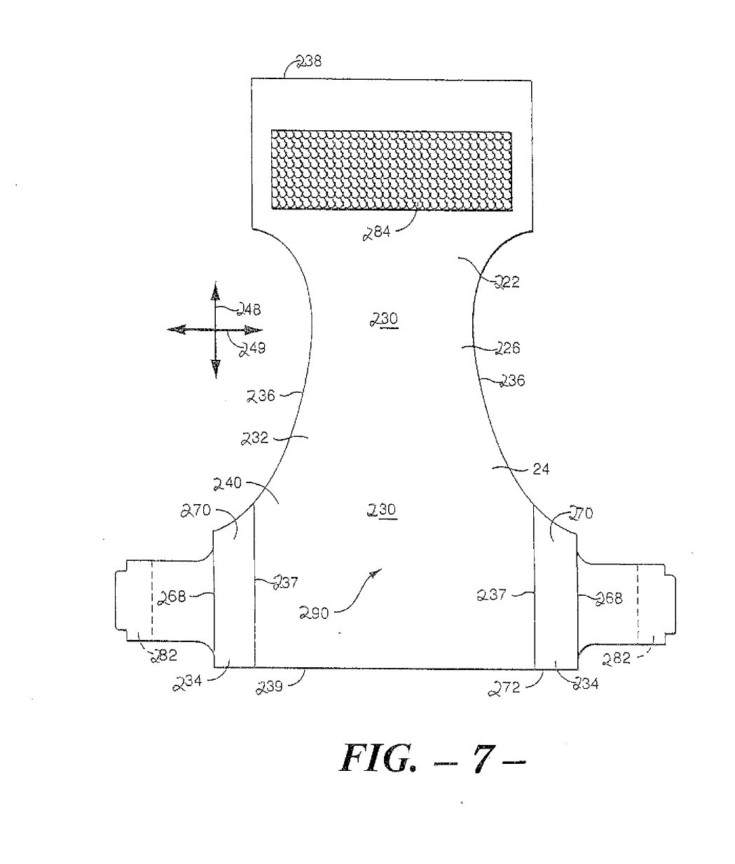

Referring to FIGS. 5 and 6 for exemplary purposes, an absorbent article 220 that may be made in accordance with the present disclosure is shown. The absorbent article 220 may or may not be disposable.

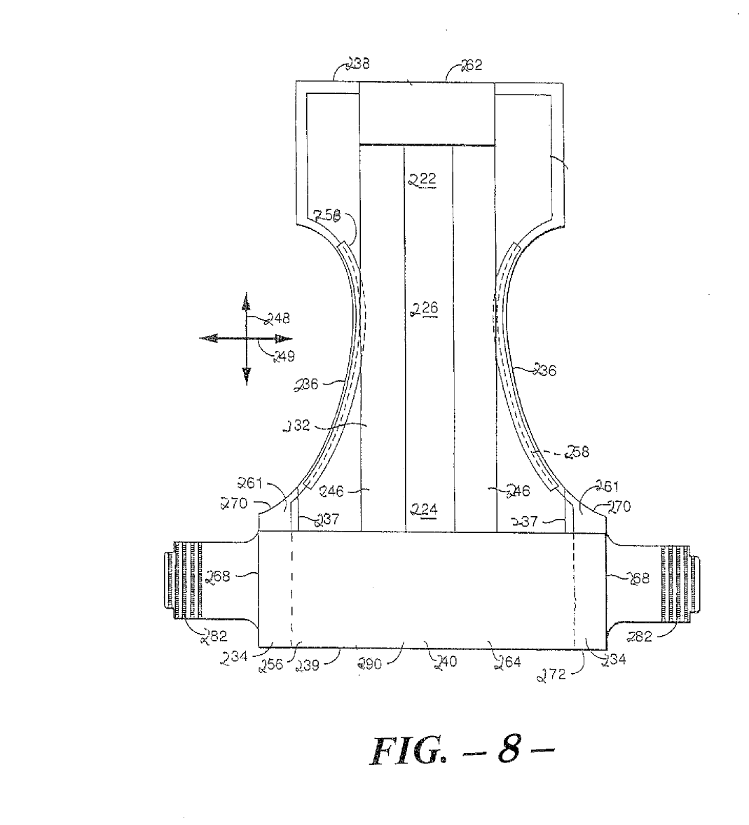

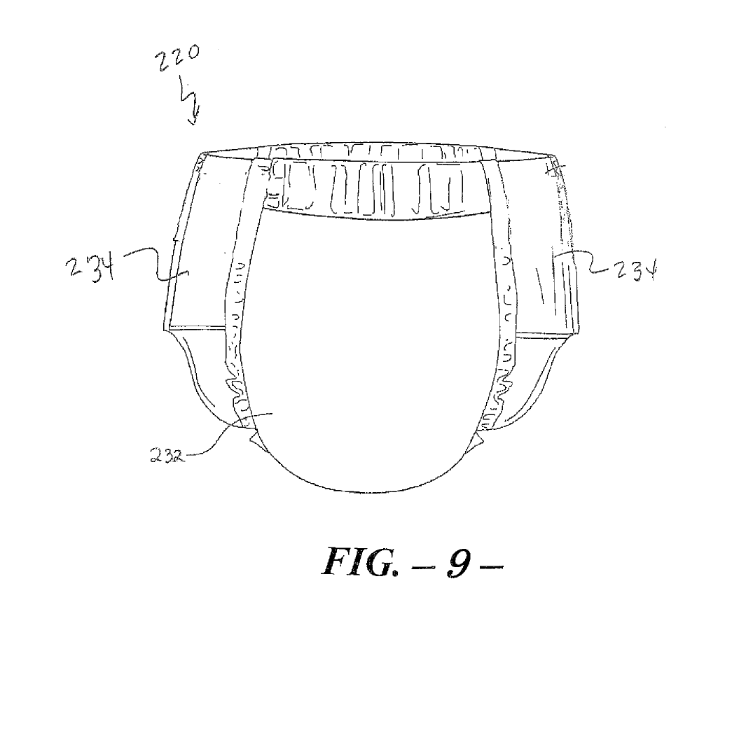

A diaper 220 is representatively illustrated in FIG. 5 in a partially fastened condition. The diaper 220 shown in FIGS. 5 and 6 is also represented in FIGS. 7 and 8 in an opened and unfolded state. Specifically, FIG. 7 is a plan view illustrating the exterior side of the diaper 220, while FIG. 8 illustrates the interior side of the diaper 220. As shown in FIGS. 7 and 8, the diaper 220 defines a longitudinal direction 248 that extends from the front of the article when worn to the back of the article. Opposite to the longitudinal direction 248 is a lateral direction 249.

The diaper 220 defines a pair of longitudinal end regions, otherwise referred to herein as a front region 222 and a back region 224, and a center region, otherwise referred to herein as a crotch region 226, extending longitudinally between and interconnecting the front and back regions 222, 224. The diaper 220 also defines an inner surface 228 adapted in use (e.g., positioned relative to the other components of the article 220) to be disposed toward the wearer, and an outer surface 230 opposite the inner surface. The front and back regions 222, 224 are those portions of the diaper 220, which when worn, wholly or partially cover or encircle the waist or mid-lower torso of the wearer. The crotch region 226 generally is that portion of the diaper 220 which, when worn, is positioned between the legs of the wearer and covers the lower torso and crotch of the wearer. The absorbent article 220 has a pair of laterally opposite side edges 236 and a pair of longitudinally opposite waist edges, respectively designated front waist edge 238 and back waist edge 239.

The illustrated diaper 220 includes a chassis 232 that, in this embodiment, encompasses the front region 222, the back region 224, and the crotch region 226. Referring to FIGS. 5-8 the chassis 232 includes an outer cover 240 and a bodyside liner 242 (FIGS. 5 and 8) that may be joined to the outer cover 240 in a superimposed relation therewith by adhesives, ultrasonic bonds, thermal bonds or other conventional techniques. Referring to FIG. 8, the liner 242 may suitably be joined to the outer cover 240 along the perimeter of the chassis 232 to form a front waist seam 262 and a back waist seam 264. As shown in FIG. 8, the liner 242 may suitably be joined to the outer cover 240 to form a pair of side seams 261 in the front region 222 and the back region 224. The liner 242 can be generally adapted, i.e., positioned relative to the other components of the article 220, to be disposed toward the wearer's skin during wear of the absorbent article. The chassis 232 may further include an absorbent structure (not shown) disposed between the outer cover 240 and the bodyside liner 242 for absorbing liquid body exudates exuded by the wearer, and may further include a pair of containment elastics or flaps 246 secured to the bodyside liner 242 for inhibiting the lateral flow of body exudates.

The elasticized containment flaps 246 as shown in FIG. 8 define a partially unattached edge which assumes an upright configuration in at least the crotch region 226 of the diaper 220 to form a seal against the wearer's body (see also FIG. 6). The containment flaps 246 can extend longitudinally along the entire length of the chassis 232 or may extend only partially along the length of the chassis. Laterally exterior to these containment flaps (also referred to in the art as BM containment flaps) can be positioned a further pair of containment elastics, which are commonly referred to as gasketing cuffs, leg gaskets or leg elastics 258. The leg elastics 258 form yet another seal about the legs of the wearer to further prevent leakage of fluids from the absorbent article 220.

As shown in FIGS. 5-8, the absorbent article 220 further includes a pair of opposing elastic side panels 234 that are attached to the back region of the chassis 232. As shown particularly in FIGS. 5 and 6, the side panels 234 may be stretched around the waist and/or hips of a wearer in order to secure the garment in place. As shown in FIGS. 7 and 8, the elastic side panels are attached to the chassis along a pair of opposing longitudinal edges 237. The side panels 234 may be attached or bonded to the chassis 232 using any suitable bonding technique. For instance, the side panels 234 may be joined to the chassis by adhesives, ultrasonic bonds, thermal bonds, or other conventional techniques.

In an alternative embodiment, the elastic side panels may also be integrally formed with the chassis 232. For instance, the side panels 234 may comprise an extension of the bodyside liner 242, of the outer cover 240, or of both the bodyside liner 242 and the outer cover 240.

In the embodiments shown in the figures, the side panels 234 are connected to the back region of the absorbent article 220 and extend over the front region of the article when securing the article in place on a user. It should be understood, however, that the side panels 234 may alternatively be connected to the front region of the article 220 and extend over the back region when the article is donned.

With the absorbent article 220 in the fastened position as partially illustrated in FIGS. 5 and 6, the elastic side panels 234 may be connected by a fastening system 280 to define a 3-dimensional diaper configuration having a waist opening 250 and a pair of leg openings 252. The waist opening 250 of the article 220 is defined by the waist edges 238 and 239 which encircle the waist of the wearer.

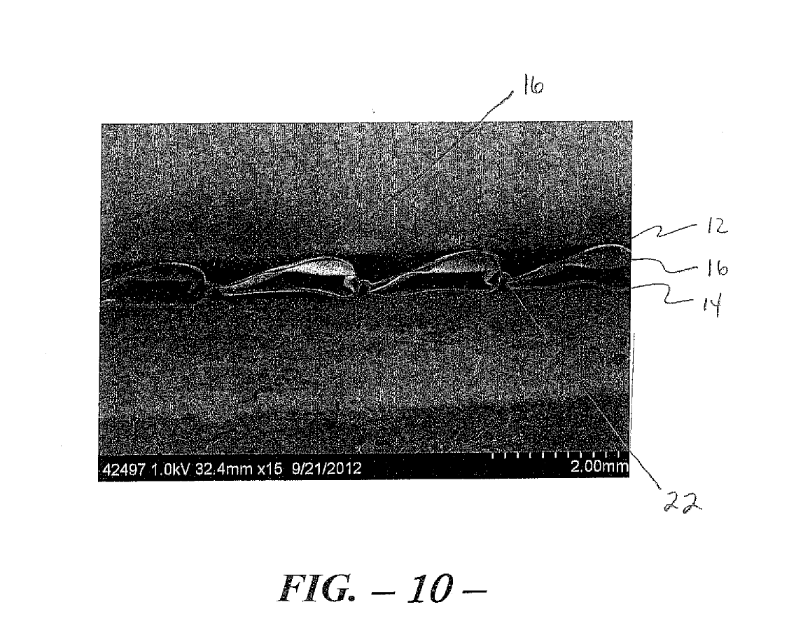

In the embodiments shown in the figures, the side panels are releasably attachable to the front region 222 of the article 220 by the fastening system. It should be understood, however, that in other embodiments the side panels may be permanently joined to the chassis 232 at each end. The side panels 234 may be permanently bonded together, for instance, when forming a diaper pant, training pant or absorbent swimwear, such as shown in FIG. 9.

The elastic side panels 234 each have a longitudinal outer edge 268, a leg end edge 270 disposed toward the longitudinal center of the diaper 220, and waist end edges 272 disposed toward a longitudinal end of the absorbent article. The leg end edges 270 of the absorbent article 220 may be suitably curved and/or angled relative to the lateral direction 249 and include the leg elastics 258 to provide a better fit around the wearer's legs. However, it is understood that only one of the leg end edges 270 may be curved or angled, such as the leg end edge of the back region 224, or alternatively, neither of the leg end edges may be curved or angled, without departing from the scope of the present disclosure. As shown in FIG. 8, the outer edges 268 are generally parallel to the longitudinal direction 248 while the waist end edges 272 are generally parallel to the transverse axis 249. It should be understood, however, that in other embodiments the outer edges 268 and/or the waist edges 272 may be slanted or curved as desired. Ultimately, the side panels 234 are generally aligned with a waist region 290 of the chassis.

The fastening system 280 may include laterally opposite first fastening components 282 adapted for refastenable engagement to corresponding second fastening components 284. In the embodiment shown in the Figures, the first fastening component 282 is located on the elastic side panels 234, while the second fastening component 284 is located on the front region 222 of the chassis 232. In one aspect, a front or outer surface of each of the fastening components 282, 284 includes a plurality of engaging elements. The engaging elements of the first fastening components 282 are adapted to repeatedly engage and disengage corresponding engaging elements of the second fastening components 284 to releasably secure the article 220 in its three-dimensional configuration.

The fastening components 282, 284 may be any refastenable fasteners suitable for absorbent articles, such as adhesive fasteners, cohesive fasteners, mechanical fasteners, or the like. In particular aspects, the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 282 include hook fasteners and the second fastening components 284 include complementary loop fasteners. Alternatively, the first fastening components 282 may include loop fasteners and the second fastening components 284 may be complementary hook fasteners. In another aspect, the fastening components 282, 284 can be interlocking similar surface fasteners, or adhesive and cohesive fastening elements such as an adhesive fastener and an adhesive-receptive landing zone or material; or the like. One skilled in the art will recognize that the shape, density and polymer composition of the hooks and loops may be selected to obtain the desired level of engagement between the fastening components 282, 284. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In the embodiment shown in the figures, the fastening components 282 are attached to the side panels 234 along the edges 268. In this embodiment, the fastening components 282 are not elastic or extendable. In other embodiments, however, the fastening components may be integral with the side panels 234. For example, the fastening components may be directly attached to the side panels 234 on a surface thereof.

As shown, the absorbent article 220 may include various extensible waist members. These extensible waist members may also be elastic for providing elasticity around the waist opening. For example, as shown in FIG. 8, the absorbent article 220 can include a front waist elastic member 254 and/or a back waist elastic member 256. The waist elastic members 254 and 256 are for providing the absorbent article with at least one form fitting property. The waist elastic members also prevent leakage of body fluids from the absorbent article.

In accordance with the present disclosure, any elastic component contained within the absorbent article 220 as shown in the figures may comprise the elastic laminate of the present disclosure. For instance, the elastic laminate of the present disclosure can be used as containment elastics such as the containment flaps 246 or leg elastics 258, the elastic side panels 234, the fastening components 282, the front elastic waist member 254, and/or the back elastic waist member 256.

The elastic laminate may also be used to produce elastic cuffs on various other garments and articles, such as surgical drapes, fenestration materials, industrial workwear, cleanroom wear, caps, surgical gowns, face masks, shoe covers and other disposable and reusable workwear and other garments. The elastic laminate may also be used in essentially any application were a gasketing cuff of some sort is needed.

The elastic laminate of the present disclosure provides various advantages when used in the above articles. For instance, elastic laminates made in accordance with the present disclosure have reduced “roll over” problems or curling problems when cut and incorporated into a garment or article.

In one embodiment, laminates can be made in accordance with the present disclosure that also have unique visual characteristics. For instance, the laminate can change visually when stretched producing an aesthetic overall look. When stretched, for instance, the lines of separation completely separate and, in some embodiments, produce areas on the laminate that have reduced opacity and/or have increased light transmission properties. For instance, especially when laminated to films, the laminate in an unstretched state may have approximately 100% opacity but, when stretched, have reduced opacity areas along the lines of separation. In one embodiment, the reduced opacity areas may be translucent or transparent. Thus, a strikingly visual appearance is created that may have aesthetic appeal.

In the embodiment described above, the elastic laminate includes a frangible layer positioned in between a pair of opposing elastic layers, such as elastic films. As shown in FIG. 1, the elastic laminate may further include one or more facing layers. The facing layer may comprise, for instance, any suitable nonwoven material, such as a meltblown web, a spunbond web, or a bonded carded web. The facing layers generally have a very low basis weight such that they do not interfere with the elastic properties of the laminate. If desired, the elastic laminate can also have multiple alternating layers of frangible layers and elastic layers to further improve durability, bulk or stretch properties. For instance, in one embodiment, the elastic laminate can include from about 2 to about 5 frangible layers, such as from about 2 to about 3 frangible layers. Each frangible layer can be positioned in between two opposing elastic layers.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An elastic laminate comprising:
a first elastic layer and a second extensible layer;
a frangible layer positioned in between the first elastic layer and the second extensible layer, the frangible layer comprising a nonwoven material, a film or a foil; and
wherein the elastic laminate has a first direction and a second direction and wherein the frangible layer includes lines of separation that extend in the first direction, the lines of separation decoupling the frangible layer from the elastic layer allowing the elastic layer to stretch and recover along the second direction.

2. An elastic laminate as defined in claim 1, wherein the second extensible layer comprises an elastic layer.

3. An elastic laminate as defined in claim 1, wherein the lines of separation define lines where the frangible layer has been at least partially severed.

4. An elastic laminate as defined in claim 1, wherein the frangible layer has an elongation at break that is less than about 75% of the elongation at break of the first elastic layer.

5. An elastic laminate as defined in claim 1, wherein the frangible layer comprises the nonwoven material, the nonwoven material comprising a spunbond web, a meltblown web, a bonded carded web, or a coform web.

6. An elastic laminate as defined in claim 1, wherein the frangible layer comprises a nonwoven material, the nonwoven material comprising a tissue web.

7. An elastic laminate as defined in claim 6, wherein the tissue web has a basis weight of from about 10 gsm to about 35 gsm.

8. An elastic laminate as defined in claim 1, wherein the frangible layer has a bulk greater than 3 cc/g.

9. An elastic laminate as defined in claim 6, wherein the tissue web includes a pattern of ridges and valleys.

10. An elastic laminate as defined in claim 1, wherein the frangible layer contains pulp fibers in an amount of at least about 50% by weight.

11. An elastic laminate as defined in claim 1, wherein the first elastic layer comprises a crosslinkable polymer.

12. An elastic laminate as defined in claim 1, wherein the first elastic layer and the second extensible layer comprise multi-layer films, each multi-layer film comprising an elastic film positioned between a first outer skin layer and a second outer skin layer.

13. An elastic laminate as defined in claim 1, wherein the first elastic layer defines perforations that extend through the elastic layer to the frangible layer.

14. An elastic laminate as defined in claim 13, wherein the frangible layer further comprises liquid absorbent particles.

15. An elastic laminate as defined in claim 1, further comprising at least one facing layer attached to the first elastic layer, the facing layer serving as an exterior surface of the laminate.

16. An elastic laminate as defined in claim 15, wherein the laminate includes a second facing layer attached to the second extensible layer.

17. An elastic laminate as defined in claim 1, wherein the frangible layer includes from about one line of separation per about 50 mm to about one line of separation per 1 mm along a direction perpendicular to the first direction.

18. An elastic laminate as defined in claim 1, wherein the frangible layer includes from about one line of separation per about 10 mm to about one line of separation per 2 mm along a direction perpendicular to the first direction.

19. An elastic laminate as defined in claim 1, wherein the laminate has a basis weight of from about 90 gsm to about 110 gsm.

20. An elastic laminate as defined in claim 1, wherein the frangible layer comprises a metal foil.

21. An absorbent article including at least one elastic member comprising the elastic laminate as defined in claim 1.

22. The absorbent article as defined in claim 21, wherein the elastic member comprises a containment elastic.

23. An absorbent article as defined in claim 21, wherein the elastic member comprises a waist elastic.

24. An absorbent article as defined in claim 21, wherein the elastic member comprises an elastic side panel or a fastening component.

25. A garment including an elastic member, the elastic member comprising the elastic laminate as defined in claim 1.

26. A garment as defined in claim 25, wherein the garment comprises an industrial garment, a clean room garment, a surgical gown, a face mask, a cap, or a shoe cover.

27. A garment as defined in claim 25, wherein the elastic member comprises an elastic cuff.

28. An elastic laminate as defined in claim 1, wherein the laminate in an unstretched state has an opacity of substantially 100% but, when stretched, has reduced opacity areas in between the lines of separation.

29. An elastic laminate as defined in claim 28, wherein the areas of reduced opacity are translucent.

30. A process for producing the elastic laminate defined in claim 1 comprising:
placing the frangible layer in between the first elastic layer and the second extensible layer to form a laminate; and
feeding the laminate in between a first roller and a second roller wherein at least one of the rollers defines grooves and wherein the laminate is fed in between the two rollers with sufficient nip pressure to form the lines of separation in the frangible layer without cutting the first elastic layer or the second elastic layer.

31. An extensible laminate comprising:
a first extensible layer and a second extensible layer;
a frangible layer positioned in between the first extensible layer and the second extensible layer, the frangible layer comprising a nonwoven material, a film or a foil; and
wherein the extensible laminate has a first direction and a second direction and wherein the frangible layer includes lines of separation that extend in the first direction, the lines of separation decoupling the frangible layer from the extensible layer allowing the extensible layer to stretch and recover along the second direction, and wherein the frangible layer is non-elastic.

32. An extensible laminate as defined in claim 31, wherein the first extensible layer and the second extensible layer are both non-elastic.
